(19) United States
(12) Patent Application Publication
(10) Pub. No.: US 2008/0146615 A1
(43) Pub. Date: Jun. 19, 2008

(54) Pharmaceutical Compositions Comprising Amorphous Benzimidazole Compounds

(75) Inventors: Indu Bhushan, Hyderabad (IN); Kavita Vermani, Hyderabad (IN); Ravinder Kodipyaka, Hyderabad (IN); Pavak Mehta, Noida (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignees: Dr. Reddy's Laboratories Limited, Hyderabad, Andhra Pradesh (IN); Dr. Reddy's Laboratories, Inc., Bridgewater, NJ (US)

(21) Appl. No.: 11/722,247

(22) PCT Filed: Dec. 20, 2005
(86) PCT No.: PCT/US2005/046393

§ 371 Date: Jun. 20, 2007

(30) Foreign Application Priority Data
Dec. 20, 2004 (IN) 1401/CHE/2004

(51) Int. Cl. A61K 31/4439; A61K 31/4184

(52) U.S. Cl. 514/338; 514/394

(57) ABSTRACT

Compositions comprising amorphous substituted benzimidazole compounds.

### INTRODUCTION TO THE INVENTION

The present invention relates to processes for the preparation of pharmaceutical compositions comprising the amorphous form of substituted benzimidazoles or their pharmaceutically acceptable salts, solvates, enantiomers or mixtures thereof, methods of use and treatment using the compositions obtained by these processes.

More specifically, the present invention relates to processes for the preparation of pharmaceutical compositions comprising the amorphous form of substituted benzimidazoles, which do not demonstrate changes in crystalline form as characterized by the X-ray diffraction (XRD) pattern of the active substance in the compositions, upon storage.

Substituted benzimidazoles are a class of compounds, finding use in a variety of gastrointestinal disorders such as gastroesophageal reflux disease (GERD), gastric ulcers, erosive esophagitis and gastritis. Molecules from the substituted benzimidazoles class of compounds that have been commercialized include omeprazole, as PRILOSEC® (a capsule dosage form for oral administration that comprises delayed release pellets of 10, 20 and 40 mg of omeprazole), omeprazole magnesium as PRILOSEC OTC (a tablet containing 20 mg omeprazole as the magnesium salt), esomeprazole magnesium as NEXIUM® (a capsule dosage form for oral administration that comprises delayed release pellets of 20 and 40 mg of the magnesium salt of the (−) enantiomer of omeprazole), lansoprazole as PREVACID® (a capsule dosage form for oral administration that comprises delayed release pellets of 15 and 30 mg of lansoprazole), pantoprazole as PROTONIX® (a delayed release tablet dosage form for oral administration of 20 and 40 mg of pantoprazole sodium), and rabeprazole as ACIPHEX® (a delayed release tablet dosage form for oral administration of 20 mg of rabeprazole sodium).

This class of compounds and certain commercially marketed specific compounds are represented by the following general structural formula:

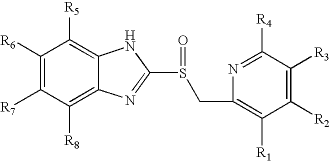

Omeprazole

| | |
|---|---|
| R₁, R₃ = | CH₃ |
| R₂, R₇ = | OCH₃ |
| R₄, R₅, R₆, R₈ = | H |

Lansoprazole

| | |
|---|---|
| R₁ = | CH₃ |
| R₂ = | OCH₂CF₃ |
| R₃, R₄, R₅, R₆, R₇, R₈ = | H |

Pantoprazole

| | |
|---|---|
| R₁, R₂ = | OCH₃ |
| R₇ = | OCF₂ |
| R₃, R₄, R₅, R₆, R₈= | H |

Rabeprazole

| | |
|---|---|
| R₁ = | CH₃ |
| R₂ = | O(CH₂)₃OCH₃ |
| R₃, R₄, R₅, R₆, R₇, R₈ = | H |

Esomeprazole

(S)- isomer of Omeprazole

Many pharmaceutical actives are known to exist in different crystalline forms. Different polymorphic forms of the same compound may have completely different properties, specially when compared with an amorphous form of the same active. Amorphous materials have properties that can be of advantage in the preparation of solid dosage forms, such as solubility/dissolution rate, bioavailability, functional mechanics and adhesivity. However, the increased reactivity of an amorphous solid, with a consequent high propensity to spontaneously transform to the crystalline state at a certain conditions such as for example relative humidity, force and temperature among others, may negatively affect the physical and chemical stability of the pharmaceutical preparation. The use of drugs and excipients in the amorphous form thus represents both a potential advantage and disadvantage to the formulator. Attempts, have therefore, been made to overcome these disadvantages by modulating the solid-state reactivity of amorphous substances, in terms of increasing or decreasing their reactivity.

Various approaches used for the formulation of an amorphous material include the use of dry granulation techniques for tableting, complexation, dry mixing, melt-extrusion, co-precipitation, spray drying, and co-milling, to name a few. Compositions comprising amorphous actives suffer from problems of form conversion either during processing or upon stability.

There has thus always been a need to produce a dosage form wherein the drug is retained in the amorphous form, either during formulation processing or during the shelf-life of the formulation.

Retaining the drug in the amorphous form in the final dosage form improves the dissolution of the final dosage form. The literature indicates that dissolution rates typically increase in the following order: pure drug substance<physical mixture<solid dispersion<melt granules<amorphous drug<tableted melt granules.

Furthermore, depending on the processing and storage conditions, amorphous forms may also absorb water from the atmosphere, which plays the role of a plasticizer, resulting in the lowering of the glass transition temperature. This phenomenon accelerates the process of crystallization. The form of the crystals hence formed is highly unpredictable. This change of the form of the drug substance affects the quality in terms of the change in the purity and identity of the dosage form. Also the presence of crystalline forms in the final composition affects the dissolution when compared to the dosage form containing the pure amorphous form of the drug in the final dosage form resulting in variability in dissolution profiles and possibly, the bioavailability of the active from the dosage form.

The various methods of preparing amorphous products known in the art include spray drying; freeze drying (lyophilisation); crash cooling from supercritical fluids, solution enhanced dispersion by supercritical fluids (SEDS); rapid expansion of supercritical solution (RESS); co-precipitation with suitable excipients (such as sugars, acids, polymers and surfactants) to form solid dispersions, molecular dispersions and co-precipitates and co-evaporates by melting or fusion or from solvents, including supercritical solvents.

It is well known that amorphous materials posses improved compression characteristics over the crystalline form. For example, commercial grades of lactose are produced by a spray drying technique to introduce some amorphous content which improves the compression force/hardness profile of the excipient (A. H. Kibbe, Handbook of Pharmaceutical Excipients, 3rd Edition, Pharmaceutical Press, page 276, 2000).

U.S. Pat. No. 6,780,435 describes a method for preparing an omeprazole pellet by applying a drug layer to an inert core wherein the drug layer consists of omeprazole, a surface active agent, a filler, a pharmaceutically acceptable alkaline agent and a binder, and coating the core with an enteric coating using solvents such as isopropyl alcohol, acetone and methylene chloride. The patent discloses the process for the preparation of the cores using a fluidized bed coater by spraying non-pareil seeds with an aqueous or non-aqueous suspension containing an alkaline agent, omeprazole, a surfactant, and a binder. The suspension medium may comprise any low viscosity solvent such as water, isopropyl alcohol, acetone, ethanol or the like. Further the patent exemplifies the use of water to form a dispersion of omeprazole along with pharmaceutically acceptable excipients.

U.S. Pat. No. 6,248,355 describes compositions of acid labile substances that do not include either alkaline reacting compounds or mannitol. The acid labile substances are omeprazole, pantoprazole, lansoprazole, leminoprazole, and praiprazole, and are not in the form of an alkaline salt. Compositions are prepared by conventional fluid bed granulation techniques and are compressed as microtablets, coated with an intermediate layer and an enteric layer, and then filled into hard gelatin capsules.

U.S. Patent Application Publication No. 2003/0104063 describes a pharmaceutical composition comprising a dispersion comprising a low-solubility drug, at least a major portion of the drug being amorphous (about 60% to 90%), and a matrix combined with a concentration-enhancing polymer. The compositions improve the stability of the drug in the dispersion, and/or the concentration of drug in a use environment.

The development of pharmaceutical compositions comprising the amorphous form of a substituted benzimidazole, which do not show change in XRD pattern of the compositions during manufacturing and upon storage would be a significant improvement in the delivery of benzimidazoles.

This and other needs are addressed by this invention.

### SUMMARY OF THE INVENTION

The present invention relates to the processes for the preparation of pharmaceutical compositions comprising the amorphous form of substituted benzimidazoles or their pharmaceutically acceptable salts, solvates, enantiomers or mixtures thereof, methods of use and treatment of different disease conditions using these compositions.

More specifically, the present invention relates to processes for the preparation of pharmaceutical compositions comprising the amorphous form of a substituted benzimidazole, which do not show change in the XRD pattern of the active substances in the compositions upon storage.

### BRIEF DESCRIPTION OF THE DRAWINGS

FIG. 1 shows XRPD patterns of omeprazole magnesium, both in the amorphous form and in the crystalline form.

FIG. 2 shows XRPD patterns of esomeprazole magnesium, both in the amorphous form and in the crystalline form.

FIG. 3 is an XRPD pattern of omeprazole magnesium in the form of a premix with povidone, as prepared in Example 1.

FIG. 4 is an XRPD pattern segment of esomeprazole magnesium in the form of a premix with meglumine and mannitol, as prepared in Example 2.

FIG. 5 is an XRPD pattern segment of omeprazole magnesium in pellets, as prepared in Example 3.

FIG. 6 is an XRPD pattern segment of placebo pellets, made using all of the excipients as in Example 3 and omitting the active ingredient.

FIG. 7 is an XRPD pattern segment of omeprazole magnesium in drug-loaded pellets, as prepared in Example 4.

FIG. 8 is an XRPD pattern segment of omeprazole magnesium in drug loaded pellets, as prepared in Example 5.

FIG. 9 is a segment of XRPD patterns of esomeprazole magnesium in delayed release pellets after manufacturing according to Example 6, and after storage at 40° C. and 75 percent relative humidity (“RH”) for 1 month.

FIG. 10 is a segment of XRPD patterns of omeprazole magnesium in delayed release pellets immediately after manufacturing according to Example 7 and after storage at 40° C. and 75 percent RH for three months.

### DETAILED DESCRIPTION

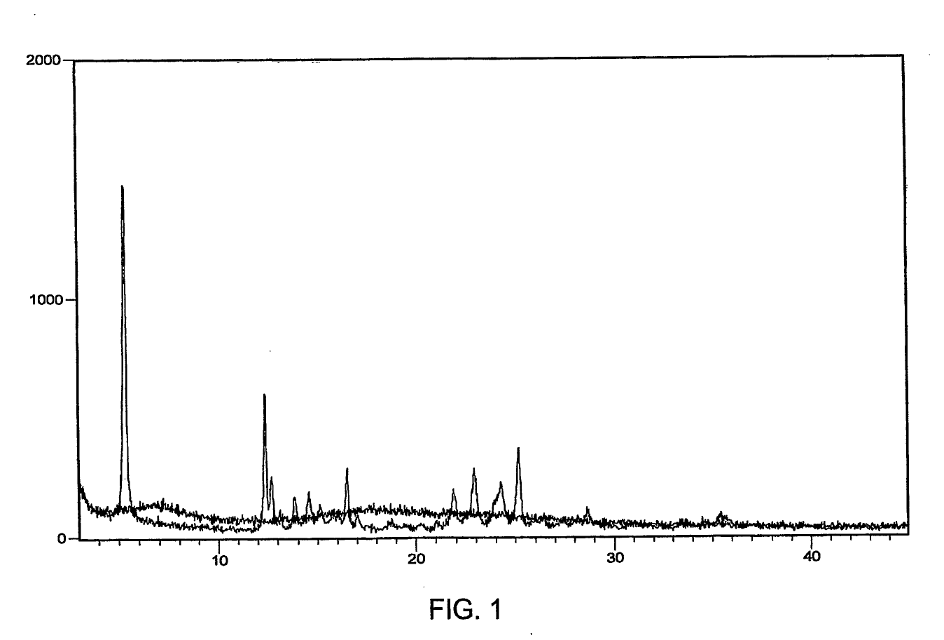

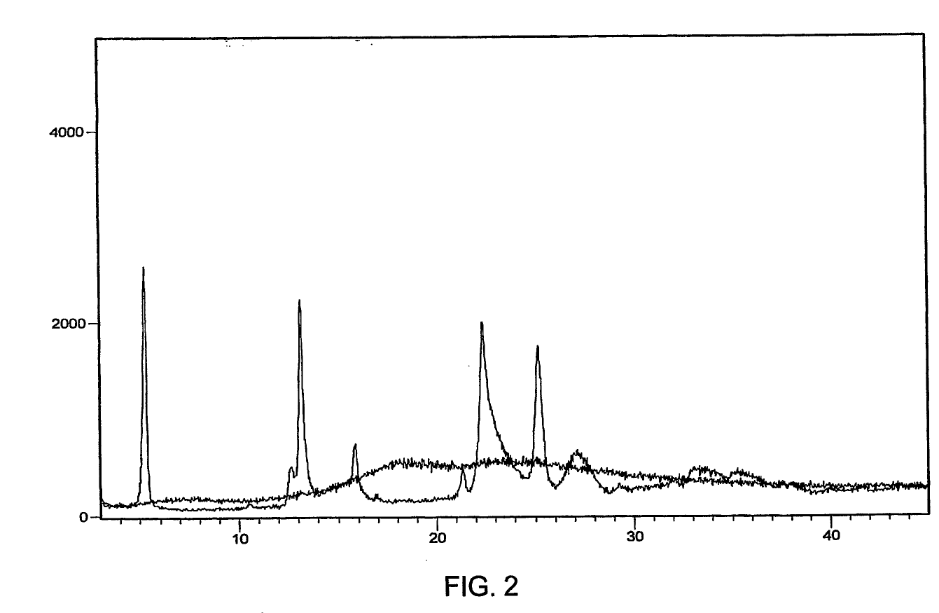

X-ray powder diffraction (“XRPD”) patterns described herein were obtained using copper K-alpha radiation (1.541 Å wavelength). In all of the figures, the vertical axis shows intensity, and the horizontal axis shows 2θ angles, in degrees. FIG. 1 is provided for references purposes, showing a diffraction pattern for the crystalline form of omeprazole magnesium having readily apparent sharp peaks, and a superimposed relatively featureless pattern for the amorphous form of the compound. Similarly, FIG. 2 shows a diffraction pattern for the crystalline form of esomeprazole magnesium having readily apparent sharp peaks, and a superimposed relatively featureless pattern for the amorphous form of that compound. In both of FIGS. 1 and 2, the crystalline forms have a strong peak about 5.3° 2θ, and the absence of a peak at or near this location can be used as an indicator of the amorphous nature of samples of the compounds.

The term “premix” herein refers to a composition prepared by dissolving or dispersing a substituted benzimidazole in an organic solvent or mixture of organic solvents with one or more pharmaceutically acceptable excipients and converting the solution or dispersion to a solid form.

The term “multi-particulate” herein refers to compositions prepared by dissolving or dispersing substituted benzimidazole in an organic solvent or mixture of organic solvents with or without a pharmaceutically acceptable excipients and depositing the solution or dispersion onto inert beads, spheres, cores, seeds, particles, or nuclei.

The present invention relates to the processes for the preparation of pharmaceutical compositions comprising an amorphous form of substituted benzimidazoles, including their pharmaceutically acceptable salts, solvates, enantiomers or mixtures thereof, and methods of use and treatment.

Solutions of a benzimidazole compound in an organic solvent or mixture of organic solvents can be converted into a solid form, with or without first being deposited onto a particulate solid substrate, such as inert beads, spheres, cores, seeds, particles, or nuclei, using solvent removal techniques such as fluidized bed drying, spray drying, vacuum drying, agitated thin film drying (ATFD) and the like, resulting in compositions wherein the substituted benzimidazole is in amorphous form. The amorphous nature of the substituted benzimidazole in the composition can remain stable during a commercially useful shelf life.

In an embodiment, a solution of a crystalline form of a substituted benzimidazole is formed in an organic solvent or mixture of organic solvents, optionally with one or more hydrophilic pharmaceutically acceptable excipients.

In another embodiment, a dispersion or solution of an amorphous form of a substituted benzimidazole is formed using an organic solvent or mixture of organic solvents, optionally with one or more pharmaceutically acceptable hydrophilic excipients.

In an embodiment, a crystalline substituted benzimidazole is taken as a starting material in solution in an organic solvent or solvent mixture, optionally with one or more pharmaceutically acceptable excipients, and processed to result in a composition comprising the substituted benzimidazole in an amorphous form.

In another embodiment, an amorphous substituted benzimidazole is taken as a starting material in a solution or dispersion in an organic solvent or solvent mixture, optionally with one or more pharmaceutically acceptable excipients, and is processed to obtain a composition comprising the substituted benzimidazole in amorphous form.

Various substituted benzimidazoles or their pharmaceutically acceptable salts, solvates, enantiomers or mixtures thereof, can be used in the present invention, including but not limited to omeprazole, lansoprazole, esomeprazole, pantoprazole, rabeprazole, leminoprazole, pariprazole, timoprazole, disulprazole and tenatoprazole.

In another embodiment of the present invention, a suitable organic solvent system comprises, but is not limited to, methanol, ethanol, 1-butanol, 2-butanol, 3-methyl-1-butanol, 1-propanol, 2-propanol, isopropanol, 1-pentanol, acetone, methyl acetate, ethyl acetate, butyl acetate, propyl acetate, isopropyl acetate, isobutyl acetate, ethyl ether, tert-butylmethyl ether, ethyl formate, chloroform, dichloromethane, and the like. The use of mixtures of solvents in various proportions is within the scope of this invention. Generally, any solvent for the benzimidazole compound can be used, provided it gives solutions having a desired solute concentration.

Various pharmaceutically acceptable hydrophilic excipients that optionally can be used in the preparation of premixes or multi particulate compositions include, but are not limited to: cellulose derivatives such as methylcellulose, carboxymethyl cellulose, hydroxypropyl methylcellulose (HPMC), cross-linked sodium carboxymethyl cellulose and hydroxypropyl cellulose; carboxymethylamide; polymers of N-vinylpyrrolidone, including copolymers and polyvinylpyrrolidone homopolymers (“povidone”); polysaccharides, sugar alcohols or polyols such as sorbitol, mannitol, xylitol, erythritol; and the like. Mixtures of excipients in various ratios as required are within the scope of this invention without limitation.

Useful inert beads, spheres, cores, seeds, particles, or nuclei can comprise water-soluble materials such as sugar spheres and the like, without limitation thereto.

The inert beads, spheres, cores, seeds, particles, or nuclei can also comprise water-insoluble materials such as: cellulose such as microcrystalline cellulose spheres, glass beads; plastic particles; water-insoluble or partially soluble inorganic materials such as calcium carbonate, dicalcium phosphate anhydrous, dicalcium phosphate monohydrate, tribasic calcium phosphate, magnesium carbonate, and magnesium oxide; and the like; without limitation thereto.

An embodiment of a process to prepare the premixes of the present invention involves:
a) providing a solution of a substituted benzimidazole in an organic solvent or mixture of organic solvents;
b) optionally, dissolving or dispersing one or more pharmaceutically acceptable hydrophilic excipients in the solution or dispersion of a); and
c) converting the solution or dispersion to a solid state by evaporating the solvent or solvent mixture, such as using a fluid bed drier, spray drier, vacuum drier, or agitated thin film drying.

An embodiment of a process to prepare multi-particulate compositions of the present invention involves:
a) providing a solution of a substituted benzimidazole in an organic solvent or mixture of organic solvents;
b) optionally, dissolving or dispersing one or more pharmaceutically acceptable hydrophilic excipient in the solution of a); and
c) depositing the solution or dispersion onto solid substrate particles; and
d) evaporating the solvent to convert the solution or dispersion to a solid state.

The premix compositions of substituted benzimidazoles prepared according to the present invention can be incorporated into pharmaceutical dosage forms, such as by filling into capsules or compressing into tablets that are optionally coated with a subcoating and/or an enteric coating using various techniques.

The multi-particulate compositions of substituted benzimidazoles that are prepared can optionally be coated with a subcoating and/or an enteric coating using techniques such pan coating, semi-automatic pan coating, or fluidized bed coating, and then can be incorporated into pharmaceutical dosage forms, such as by filling into capsules or compressing into tablets, which can then be further coated, as desired.

Compositions containing the amorphous substituted benzimidazoles of the invention typically will be formulated into dosage forms, in combination with one or more pharmaceutical excipients.

Tablets can be prepared using direct compression by mixing directly compressible excipients with the premix composition or multi-particulate compositions of substituted benzimidazoles. The blend so obtained can be compressed using suitable tablet tooling with the help of rotary tablet presses.

Tablets can be prepared using wet granulation, wherein excipients are granulated, dried, milled and sifted to get a desired particle size and blended with a premix composition or multi-particulate compositions of substituted benzimidazoles, with or without desired pharmaceutical excipients such as disintegrants, glidants, lubricants, and colorants. The blend so obtained can be compressed using suitable tooling with equipment such as rotary tablet presses, or other equipment as will be apparent to those skilled in the art.

Pharmaceutical dosage forms of the present invention may contain one or more diluents to increase the final composition mass so that it becomes easier for the patient and the caregiver to handle.

Common diluents that can be used in pharmaceutical dosage forms comprise, but are not limited to, any of microcrystalline cellulose (MCC), silicified MCC (e.g. PROSOLV™ HD 90), microfine cellulose, lactose, starch, pregelatinized starch, sugar, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide, and the like.

The pharmaceutical dosage forms may further include a disintegrant. Useful disintegrants include but are not limited to carboxymethyl cellulose calcium, carboxymethyl cellulose sodium (e.g. Ac-Di-Sol®, Primellose®), crospovidone (e.g. Kollidon®, Polyplasdone®), povidone K-30, polacrilin potassium, starch, pregelatinized starch, and sodium starch glycolate (e.g. Explotab®).

In an embodiment, pharmaceutical dosage forms optionally include one or more surfactants such as anionic, cationic, and nonionic surfactants. These include, but are not limited to: anionic surfactants such as chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, and sodium dodecyl sulfate (SDS); cationic surfactants such as cetylpyridinium chloride monohydrate and hexadecyltrimethylammonium bromide; nonionic surfactants such as N-decanoyl-N-methylglucamine, octyl a-D-glucopyranoside, n-Dodecyl b-D-maltoside (DDM), and polyoxyethylene sorbitan esters like polysorbates; and the like.

Stabilizers that can be used in this invention include, are but not limited to, oxides such as magnesium oxide, calcium oxide, silicon dioxide, amines such as TRIS (tromethamine), ethanolamine, diethanolamine, triethanolamine, N-methyl-glucamine (meglumine), glucosamine, ethylenediamine, diethylamine, triethylamine, isopropylamine, diisopropylamine, urea, and alkaline amino acids such as L-arginine, cysteine, tyrosine, histidine, and lysine.

Pharmaceutical dosage forms may further include other excipients, such as but not limited to, pharmaceutically acceptable glidants, lubricants, opacifiers, colorants and other commonly used excipients.

In an embodiment, the dosage forms of said invention optionally are provided with a final film coating.

In an embodiment, a suitable solvent system such as aqueous, alcoholic, hydro-alcoholic, or organic may be used for film coating.

In yet another embodiment, a suitable solvent system for the coating comprises solvents such as, but not limited to, water, ethanol, isopropanol, acetone, methylene chloride, and the like.

Plasticizers can be added to a polymeric dispersion to make it more flexible and less brittle by reducing the glass transition temperature of the polymer. Suitable plasticizers include, but are not limited to: organic esters such as phthalate esters (diethyl, dibutyl), dibutyl sebacate, citrate esters (triethyl, acetyl triethyl, acetyl tributyl) and triacetin; oils and glycerides such as castor oil, acetylated mono glycerides, fractionated coconut oil, stearic and palmitic acid, isopropyl myristate, glycols, glyceryl monostearate, chlorobutanol, benzyl benzoate; and the like. Any plasticizer is acceptable as long as it plasticizes the polymer and is compatible with all components of the composition. Of course, it is to be understood that the plasticizer should be biocompatible and nontoxic.

Pharmaceutical compositions of the invention comprising an amorphous form of substituted benzimidazoles are used in the treatment of a variety of gastrointestinal disorders such as gastroesophageal reflux disease (GERD), gastric ulcers, erosive esophagitis, and gastritis.

In general, the formation of an amorphous benzimidazole premix should proceed in the substantial absence of water. If an amorphous benzimidazole compound is combined with an excipient in an aqueous environment, and then coated onto a solid substrate, a significant portion of the benzimidazole compound frequently will be present in a crystalline form, in the final composition. However, after an amorphous benzimidazole compound coating has been applied to a substrate, subsequent coatings that are applied can have an aqueous content.

The following examples will further illustrate certain aspects and embodiments of the invention in greater detail and are not intended to limit the scope of the invention.

### EXAMPLE 1

### Premix of Amorphous Omeprazole Magnesium with Povidone

| Ingredients | Quantity (mg) |
|---|---|
| Omeprazole magnesium | 10 |
| (amorphous) | |
| Polyvinylpyrrolidone (Povidone K | 10 |
| 30) | |
| Methanol | 60 |

### Manufacturing Process:

1. Omeprazole magnesium and povidone K 30 were dissolved in methanol.
2. The solution of step 1 was dried in a rotary evaporator (Laborota 4000, Heidolph Instruments GmbH & Co. KG, Schwabach, Germany) at 40° C. under vacuum (15 to 25 mm Hg).

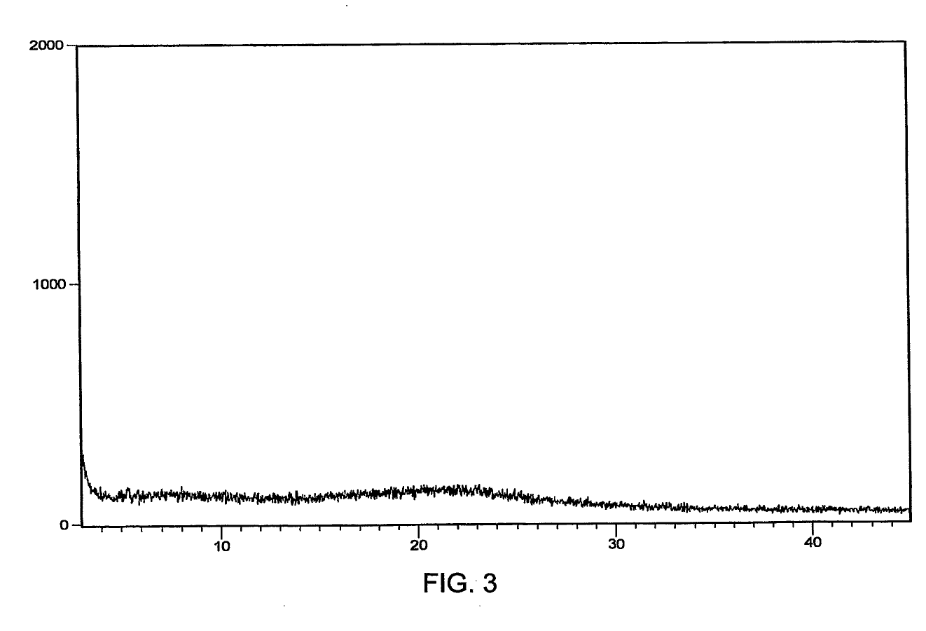

The XRPD pattern of the omeprazole magnesium premix (FIG. 3) did not include any significant crystalline peaks.

### EXAMPLE 2

### Premix of Amorphous Esomeprazole Magnesium with Meglumine and Mannitol

| Ingredients | Quantity (mg) |
|---|---|
| Esomeprazole magnesium | 40 |
| (amorphous) | |
| Mannitol | 37 |
| Meglumine | 3 |
| Methanol | 200 |

### Manufacturing Process:

1. Esomeprazole magnesium was dissolved in methanol, then mannitol and meglumine were dispersed in the solution.
2. The resulting dispersion was spray dried using a Buchi mini spray drier, Model D-191, with an inlet air temperature of 40° C., outlet air temperature of 25-27° C. and a spray rate of 7-10%.

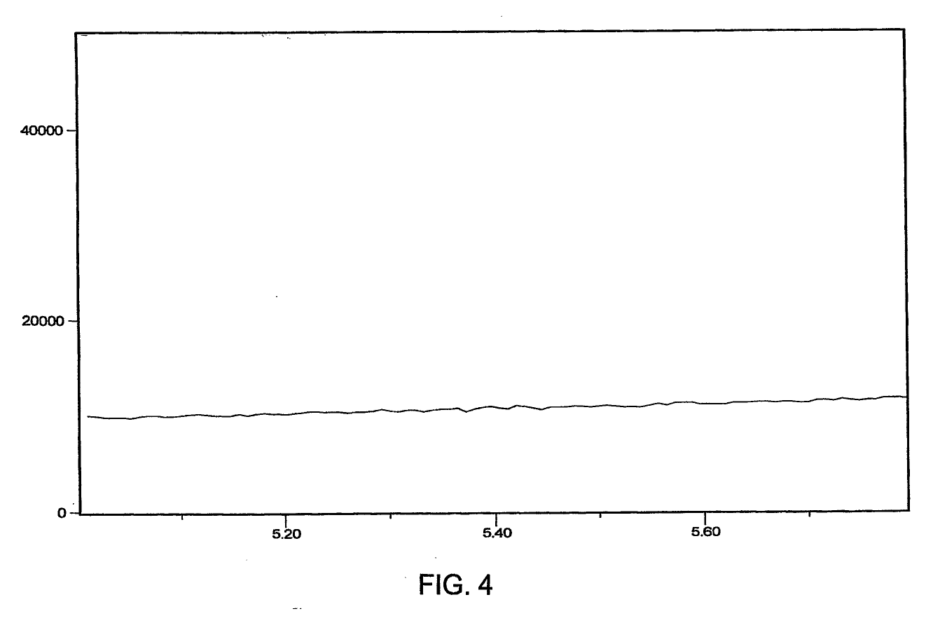

The XRPD pattern segment for the premix prepared by spray drying (FIG. 4) does not show a characteristic peak of crystalline esomeprazole magnesium.

### EXAMPLE 3

### Enteric Coated Multi-Particulate Composition Prepared Using Amorphous Omeprazole Magnesium and Microcrystalline Cellulose Spheres

| | |
|---|---|
| Ingredients | Quantity (mg) |

Drug loaded pellets

| | |
|---|---|
| Omeprazole magnesium (amorphous) | 72.1 |
| Microcrystalline cellulose spheres | 300 |
| (CELPHERE ™ CP 203)* | |
| Polyvinylpyrrolidone (Povidone K 30) | 72.1 |
| Magnesium oxide | 61.25 |
| Methanol | 400 |

Subcoating composition

| | |
|---|---|
| Drug loaded pellets | 300 |
| Zein | 24.86 |
| Methacrylic acid copolymer type C^{#} | 3.95 |
| Triethyl citrate | 0.4 |
| Isopropyl alcohol | 237 |
| Water | 26 |

Enteric coating composition

| | |
|---|---|
| Subcoated pellets | 300 |
| Methacrylic acid copolymer type C^{#} | 183.8 |
| Triethyl citrate | 18.4 |
| Glyceryl monostearate | 3.9 |
| Titanium oxide | 3.9 |
| Isopropyl alcohol | 2100 |

*CELPHERE ™ CP 203 is a product of Asahi Kasei Chemicals Corporation, Tokyo, Japan, having 150-300 μm particle sizes.
^{#}Methacrylic acid copolymer type C is EUDRAGIT ™ L 100 55 manufactured by Röhm GmbH & Co. KG, Darmstadt, Germany

### Manufacturing Process:

1. Povidone K 30 was dissolved in methanol.
2. Magnesium oxide was dispersed in the solution of step 1.
3. Omeprazole magnesium was dissolved in the dispersion of step 2.
4. Above dispersion was maintained at a temperature of 2-8° C. and loaded onto CELPHERE™ CP 203 using a fluidized bed processor with bottom spray and the following process parameters:

| | |
|---|---|
| Inlet air temperature | 35-45° C. |
| Product temperature | 26-32° C. |
| Exhaust rpm | 600-800 rpm |
| Atomization air pressure | 1.6-1.8 kg/cm² |
| Spray rate | 5-8 g/minute |

5. Drug loaded pellets thus obtained were further sub-coated with a solution of zein, triethyl citrate, and methacrylic acid copolymer type C in aqueous isopropanol.
6. After subcoating, the pellets were enteric coated using a dispersion of methacrylic acid copolymer type C, triethyl citrate, glyceryl monostearate, and titanium oxide in isopropanol.

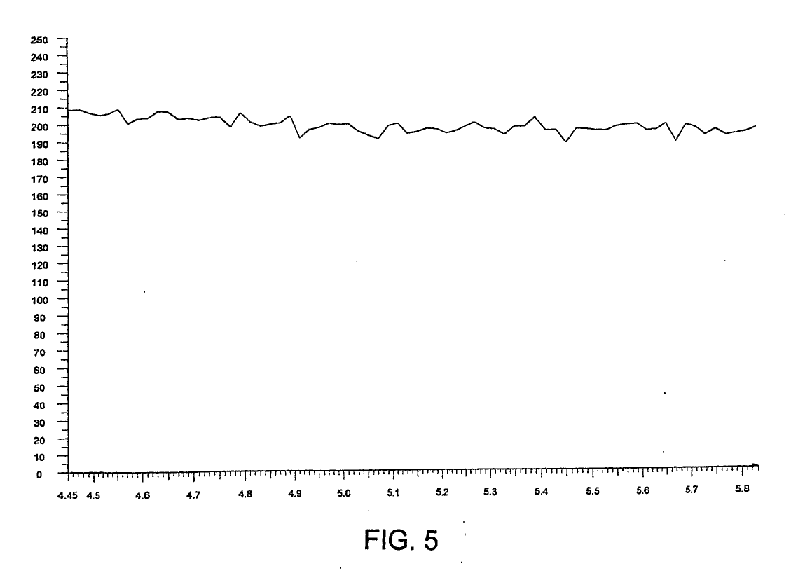

The XRPD pattern segment for the multi-particulate composition after drug layering onto microcrystalline cellulose (FIG. 5) does not show a characteristic peak for crystalline omeprazole magnesium.

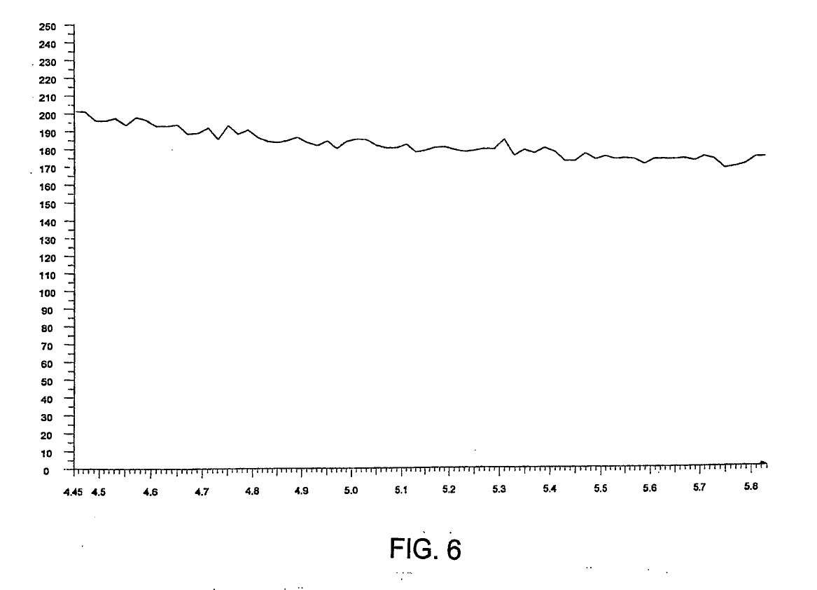

This experiment was repeated, omitting any esomeprazole magnesium, and the XRPD pattern segment of the pellets that were obtained is FIG. 6.

### EXAMPLE 4

### Multi Particulate Composition Prepared Using Amorphous Omeprazole Magnesium

| | |
|---|---|
| Ingredients | Quantity (mg) |

Drug loaded pellets

| | |
|---|---|
| Omeprazole magnesium (amorphous) | 72.1 |
| Microcrystalline cellulose spheres | 300 |
| (CELPHERE ™ CP 203) | |
| Polyvinylpyrrolidone (Povidone K 30) | 72.1 |
| Magnesium oxide | 61.25 |
| Methanol | 400 |

### Manufacturing Process:

7. Povidone K 30 was dissolved in methanol.
8. Magnesium oxide was dispersed in the solution of step 1.
9. Omeprazole magnesium was dissolved in the dispersion of step 2.
10. Above dispersion was maintained at a temperature of 16-22° C. and loaded onto microcrystalline cellulose spheres using a fluidized bed processor with bottom spray and the following process parameters:

| | |
|---|---|
| Inlet air temperature | 35-45° C. |
| Product temperature | 26-32° C. |
| Exhaust rpm | 600-800 rpm |
| Atomization air pressure | 1.6-1.8 kg/cm² |
| Spray rate | 5-8 g/minute |

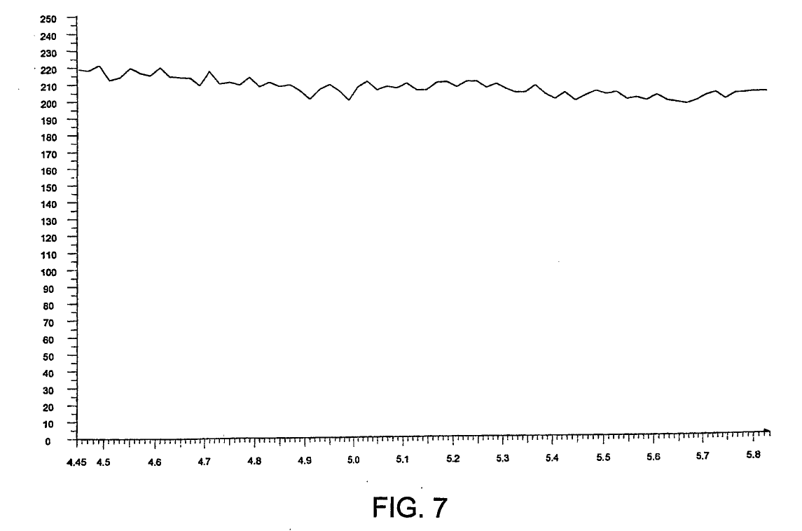

The XRPD pattern segment of the multi-particulate compositions of sugar spheres (FIG. 7) does not show a characteristic peak of crystalline omeprazole magnesium.

### EXAMPLE 5

### Multi-Particulate Composition Prepared Using Crystalline Omeprazole Magnesium

| Ingredients | Quantity (mg) |
|---|---|
| Omeprazole magnesium (crystalline) | 66.6 |
| Microcrystalline cellulose spheres | 300 |
| (CELPHERE ™ CP 203) | |
| Polyvinylpyrrolidone (Povidone K 30) | 66.6 |
| Magnesium oxide | 56.6 |
| Methanol | 1315 |

### Manufacturing Process:

1. Povidone K 30 was dissolved in methanol and magnesium oxide was dispersed in the solution.
2. Omeprazole magnesium was dissolved in the dispersion of step 1.
3. The above dispersion was maintained at a temperature of 2-8° C. and loaded onto CELPHERE™ CP 203 using a fluidized bed processor.

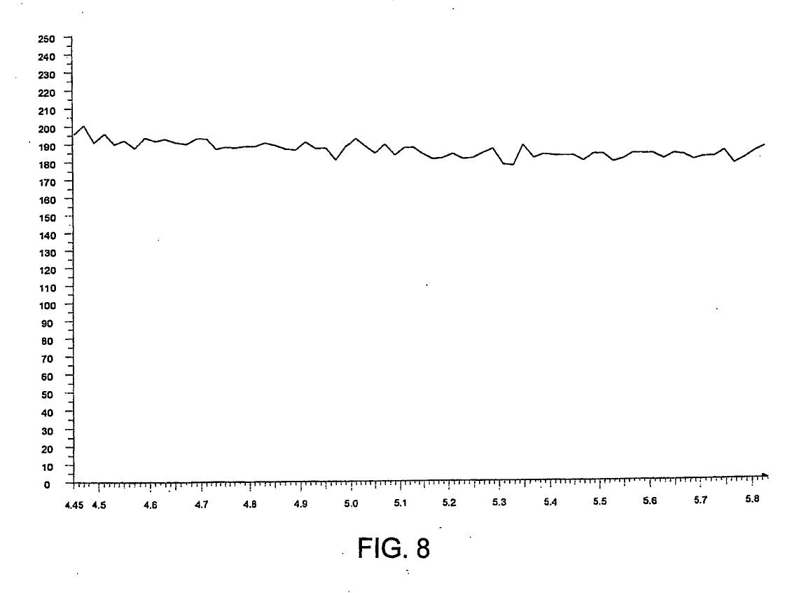

The XRPD pattern segment of the multi-particulate compositions (FIG. 8) does not show a characteristic peak of crystalline omeprazole magnesium.

### EXAMPLE 6

### Enteric Coated Multi Particulate Compositions of Esomeprazole Magnesium

| | |
|---|---|
| Ingredients | Quantity (mg) |

Drug layer composition

| | |
|---|---|
| Esomeprazole magnesium | 41.4 |
| (amorphous) | |
| Sugar spheres (40/60 mesh fraction) | 30 |
| PLASDONE ™ S 630* | 40 |
| Magnesium oxide | 20 |
| Methanol | q.s. |

Subcoating composition

| | |
|---|---|
| Hydroxypropyl methylcellulose, 5 cPs | 29.9 |
| Polyethylene glycol 6000 (PEG 6000) | 3 |
| Isopropyl alcohol | q.s. |
| Dichloromethane | q.s. |

Enteric coating composition

| | |
|---|---|
| Methacrylic acid copolymer type C | 128 |
| Triethyl citrate | 32 |
| Talc | 25.6 |
| Polysorbate 80 | 1.3 |
| Glyceryl monostearate | 6.4 |
| Water | q.s. |

Overcoating composition

| | |
|---|---|
| Hydroxypropylmethyl cellulose, 5 cPs | 20.3 |
| Polyethylene glycol 6000 | 2.03 |
| Talc | 4.06 |
| Magnesium stearate | 0.8 |
| Isopropyl alcohol | q.s. |
| Dichloromethane | q.s. |

*PLASDONE ™ S 630 Copovidone is a copolymer of N-vinyl-2-pyrrolidone and vinyl acetate and is manufactured by ISP Corporation, Japan.

### Manufacturing Process:

1. Plasdone S630 was dissolved in methanol.
2. Magnesium oxide was dispersed in the solution of step 1.
3. Esomeprazole magnesium was dissolved in the dispersion of step 2.
4. The above dispersion was maintained at a temperature of 2-8° C. and loaded onto sugar spheres using a fluidized bed processor with bottom spray and with following process parameters:

| | |
|---|---|
| Inlet air temperature | 44-47° C. |
| Product temperature | 30-32° C. |
| Exhaust RPM | 600-800 RPM |
| Atomization air pressure | 1.8-2.0 kg/cm² |
| Spray rate | 5-8 g/minute |

5. Drug loaded pellets thus obtained were further sub-coated with a solution of hydroxypropyl methylcellulose and PEG 6000 in isopropyl alcohol and dichloromethane.
6. After subcoating, the pellets were enteric coated using an aqueous dispersion of methacrylic acid copolymer type C containing triethyl citrate, glyceryl monostearate, talc, and polysorbate 80 followed by an overcoating with hydroxypropyl methylcellulose.

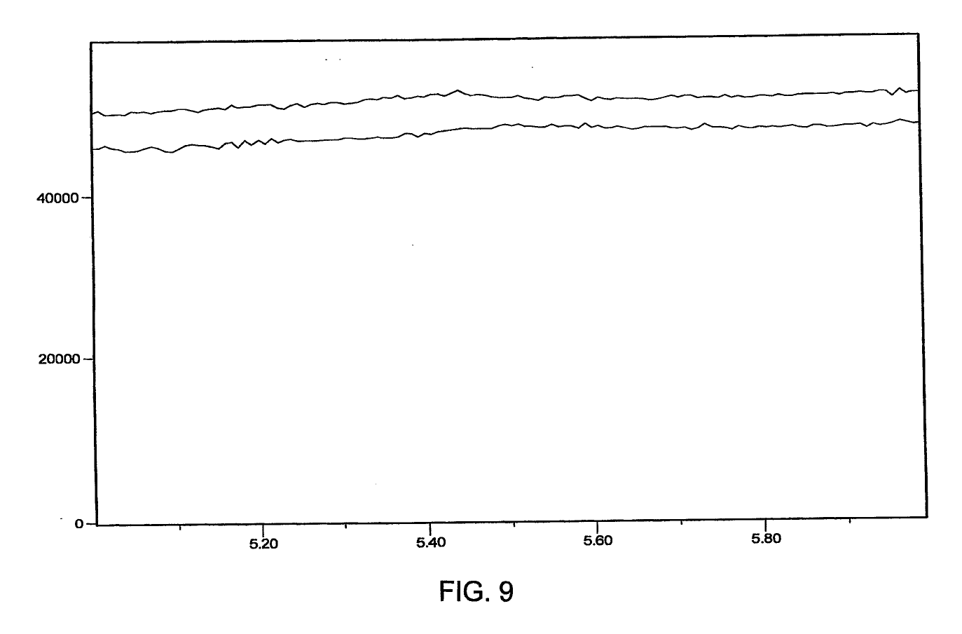

XRPD pattern segments of the pellets are shown in FIG. 9, as obtained promptly after manufacturing (upper pattern) and after one month of storage in closed high density polyethylene (“HDPE”) containers at 40° C. and 75 percent RH (lower pattern). The pattern segments do not show a characteristic peak of crystalline esomeprazole magnesium.

### EXAMPLE 7

### Enteric Coated Multi-Particulate Composition of Omeprazole Magnesium

| | |
|---|---|
| Ingredients | Quantity (mg) |

Drug layer composition

| | |
|---|---|
| Omeprazole magnesium (amorphous) | 20.6 |
| Sugar spheres (60/80 mesh fraction) | 7 |
| Hydroxypropyl methylcellulose, 5 cPs | 8 |
| Meglumine | 0.5 |
| Methanol | q.s. |

Subcoating composition

| | |
|---|---|
| Hydroxypropyl methylcellulose, 5 cPs | 3.25 |
| Talc | 6.03 |
| Magnesium stearate | 0.5 |
| Isopropanol | q.s. |
| Dichloromethane | q.s. |

Enteric coating composition

| | |
|---|---|
| Methacrylic acid copolymer type C | 46.12 |
| Triethyl citrate | 5.77 |
| Talc | 2.88 |
| Glyceryl monostearate | 1.73 |
| Titanium dioxide | 1.15 |
| Isopropyl alcohol | q.s. |

### Manufacturing Process:

1. Hydroxypropyl methylcellulose, 5 cPs was dissolved in a mixture of methanol and dichloromethane and meglumine was added.
2. Omeprazole magnesium (amorphous) was dissolved in the preparation of step 1.
3. The above preparation of step 2 was maintained at a temperature of 2-8° C. and loaded onto sugar spheres using a fluidized bed processor with bottom spray and the following process parameters:

| | |
|---|---|
| Inlet air temperature | 45-55° C. |
| Product temperature | 28-29° C. |
| Exhaust RPM | 600-800 RPM |
| Atomization air pressure | 1.8-2.0 kg/cm² |
| Spray rate | 5-8 g/minute |

4. Drug-loaded pellets thus obtained were further subcoated with a dispersion of hydroxypropyl methylcellulose 5 cPs, talc, and magnesium stearate in isopropanol and dichloromethane.
5. After subcoating, the pellets were enteric coated using a non-aqueous dispersion of methacrylic acid copolymer type C containing triethyl citrate, glyceryl monostearate, and talc in isopropyl alcohol.

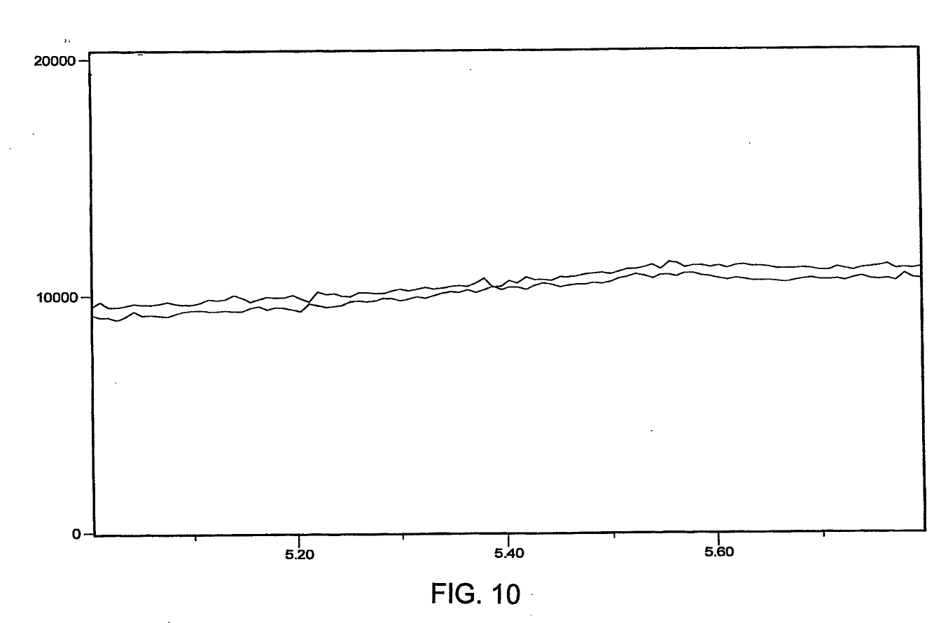

XRPD pattern segments of the pellets are shown in FIG. 10, as obtained promptly after manufacturing (upper pattern) and after three months of storage in closed HDPE containers at 40° C. and 75 percent RH (lower pattern). The pattern segments do not show a characteristic peak of crystalline esomeprazole magnesium.

1. A process for preparing an amorphous benzimidazole composition, comprising:
providing a solution of a substituted benzimidazole in an organic solvent;
optionally, dissolving or dispersing one or more hydrophilic excipients in the solution; and
removing solvent.

2. The process of claim 1, wherein a substituted benzimidazole comprises at least one of omeprazole, lansoprazole, esomeprazole, pantoprazole, rabeprazole, leminoprazole, pariprazole, timoprazole, disulprazole and tenatoprazole, or a salt of any of the foregoing.

3. The process of claim 1, wherein a substituted benzimidazole comprises omeprazole or a salt thereof.

4. The process of claim 1, wherein a substituted benzimidazole comprises esomeprazole or a salt thereof.

5. The process of claim 1, wherein a hydrophilic excipient is dissolved or dispersed in the solution.

6. The process of claim 3, wherein a hydrophilic excipient comprises at least one of: a cellulose derivative; carboxymethylamide; a polymer of N-vinylpyrrolidone; a polysaccharide; a sugar alcohol; and a polyol.

7. The process of claim 1, wherein a solution or dispersion is deposited onto a particulate solid substrate, before solvent is removed.

8. The process of claim 7, wherein a solid substrate comprises particles of microcrystalline cellulose, sugar, glass; plastic, or water-insoluble or partially water-soluble inorganic material.

9. The process of claim 1, wherein a solution or dispersion is deposited onto inert beads, spheres, cores, seeds, particles, or nuclei, before solvent is removed.

10. The process of claim 1, wherein solvent is removed by fluidized bed drying, spray drying, vacuum drying, or agitated thin film drying.

11. An amorphous benzimidazole composition prepared by the process of claim 1.

12. An amorphous benzimidazole composition prepared by the process of claim 1, comprising at least one of omeprazole, lansoprazole, esomeprazole, pantoprazole, rabeprazole, leminoprazole, pariprazole, timoprazole, disulprazole and tenatoprazole, or a salt of any of the foregoing.

13. An amorphous benzimidazole composition prepared by the process of claim 1, containing a hydrophilic excipient comprising at least one of: a cellulose derivative; carboxymethylamide; a polymer of N-vinylpyrrolidone; a polysaccharide; a sugar alcohol; and a polyol.

14. An amorphous benzimidazole composition prepared by the process of claim 1, comprising omeprazole, esomeprazole, or a mixture thereof, and a hydrophilic excipient comprising at least one of: a cellulose derivative; carboxymethylamide; a polymer of N-vinylpyrrolidone; a polysaccharide; a sugar alcohol; and a polyol.

15. An amorphous benzimidazole composition prepared by the process of claim 7, comprising at least one of omeprazole, lansoprazole, esomeprazole, pantoprazole, rabeprazole, leminoprazole, pariprazole, timoprazole, disulprazole and tenatoprazole, or a salt of any of the foregoing.

16. An amorphous benzimidazole composition prepared by the process of claim 7, containing a hydrophilic excipient comprising at least one of: a cellulose derivative; carboxymethylamide; a polymer of N-vinylpyrrolidone; a polysaccharide; a sugar alcohol; and a polyol.

17. An amorphous benzimidazole composition prepared by the process of claim 7, comprising omeprazole, esomeprazole, or a mixture thereof, and a hydrophilic excipient comprising at least one of: a cellulose derivative; carboxymethylamide; a polymer of N-vinylpyrrolidone; a polysaccharide; a sugar alcohol; and a polyol.

18. An amorphous benzimidazole composition prepared by the process of claim 17, wherein a solid support comprises particles of microcrystalline cellulose, sugar, glass; plastic, or water-insoluble or partially water-soluble inorganic material.

19. A pharmaceutical dosage form, comprising an amorphous benzimidazole composition prepared by the process of claim 1 and at least one pharmaceutical excipient.

20. A pharmaceutical dosage form, comprising an amorphous benzimidazole composition prepared by the process of claim 7 and at least one pharmaceutical excipient.
